(19) United States
(12) Patent Application Publication
(10) Pub. No.: US 2024/0032670 A1
(43) Pub. Date: Feb. 1, 2024

(54) PATCH FOR BRINGING A COSMETIC COMPOSITION INTO CONTACT WITH THE SKIN

(71) Applicant: L'OREAL, Paris (FR)

(72) Inventor: Eric CAULIER, CLICHY (FR)

(21) Appl. No.: 18/256,067

(22) PCT Filed: Dec. 14, 2021
(86) PCT No.: PCT/EP2021/085760

§ 371 Date: Jun. 6, 2023

(30) Foreign Application Priority Data
Dec. 17, 2020 (FR) 2013497

(51) Int. Cl. A45D 34/04; A45D 40/26

(52) U.S. Cl. CPC A45D 34/04; A45D 40/26; A45D 2200/1036; A45D 2200/25

(57) ABSTRACT

The patch (1) comprises a substantially planar main body (2), having an application face (3) adapted to be attached to the skin (P) in a repositionable fashion by means of an attachment layer (4) comprising an adhesive, the main body (2) having a blind cavity (6) adapted to directly receive the cosmetic composition (C),

the main body (2) consisting of overlaid layers comprising:
at least one layer (10) for receiving a cosmetic composition (C), the receiving layer (10) delimiting the bottom of the cavity,
at least one intermediate layer (12) inserted between the receiving layer (10) and the attachment layer (4), the intermediate layer (12) delimiting the periphery of the cavity (6), and defining, at the periphery of the cavity (6), the application face (3) receiving the attachment layer (4).

This invention relates to a patch for bringing a cosmetic composition into contact with the skin, comprising a substantially planar main body, having an application face adapted to be attached to the skin in a repositionable fashion by means of an attachment layer comprising at least one adhesive, the main body having a blind cavity defining a bottom and opening via an opening onto the application face, said cavity being adapted to directly receive the cosmetic composition to be brought into contact with the skin, and a peripheral attachment surface being formed on the application face about the cavity, the attachment layer extending over the peripheral attachment surface of the application face of the main body. The term “patch” denotes here, and as commonly defined, a self-adhesive dermal device which enables the diffusion of a substance on and/or through the skin.

The cosmetic composition comprises for example a cosmetic body surface care, coloring or/and makeup product.

More generally, a cosmetic product is as defined in EC Regulation No. 1223/2009 of the European Parliament and the Council of Nov. 30, 2009, relating to cosmetic products.

A patch of the type cited above, comprising a plurality of subcavities and wherein the main body and the attachment layer are made of silicone and are mounted by molding or co-molding is known from FR3082723A1.

Such a patch has the advantages of boosting the effectiveness of the cosmetic composition placed in the different blind cavities and applied against the skin for a variable application time, and also of being washable and reusable.

However, the design of such a patch is complex and costly, and problems of silicone adhesion to the mold may be encountered during the manufacture of the patch, thus generating products failing to meet the patch specifications.

An aim of the invention is that of remedying the drawbacks cited above by providing a patch which is simple to use, while retaining the effectiveness thereof by supplying a maximum amount of cosmetic composition on a very specific area to be treated.

To this end, the invention relates to a patch of the type cited above, wherein the main body consists of overlaid layers, the overlaid layers comprising:
at least one layer for receiving a cosmetic composition, the receiving layer delimiting the bottom of the cavity,
at least one intermediate layer inserted between the layer for receiving a cosmetic composition and the attachment layer, the intermediate layer delimiting the periphery of the cavity, and defining, at the periphery of the cavity, the application face receiving the attachment layer.

Such a patch helps boost the effectiveness of the cosmetic product. The action is concentrated on the surface whereon the patch is applied, by creating at least partial occlusivity, favoring opening of the skin pores and thus accelerating the penetration of the cosmetic composition.

The attachment layer is used to position and reposition the patch at will on the desired skin area. The patch can be washed and refilled once again with cosmetic composition for later use.

The intermediate layer acts as a spacer and the thickness thereof determines the maximum volume of cosmetic composition that can be filled in the cavity, thus helping manage the treatment time.

The overlaying of the layers is very simple to implement, quick and inexpensive.

According to an alternative embodiment, the main body further comprises a binding layer inserted between the intermediate layer and the receiving layer. The binding layer makes it possible to protect the intermediate layer, in particular so that the material of the receiving layer does not penetrate the material of the intermediate layer.

According to another alternative embodiment, the intermediate layer is made of foam.

Such a material provides the user with comfort and the patch with flexibility, and absorbs the deformation in a transverse direction to the thickness of the patch.

According to an advantageous alternative embodiment, the attachment layer comprises a biocompatible adhesive intended to be applied on the skin. Such an adhesive particularly ensures a satisfactory hold over time, as well as a good ability to reposition the patch on the skin.

According to another alternative embodiment, the attachment layer includes an adhesive on the face thereof in contact with the intermediate layer, the adhesive having a greater adherence than the adherence of the attachment layer on the application face thereof opposite the intermediate layer. In this way, the attachment layer is not detached from the patch when the patch is removed after use, and leaves no residue on the skin.

According to another alternative embodiment, at least one adhesive of the attachment layer has a mass density between 8 g/m² and 300 g/m². Preferably, the adhesive of the attachment layer comprises or consists of silicone, in particular a silicone gel. In this embodiment, the adhesive of the attachment layer has a mass density between 20 g/m² and 300 g/m², preferably between 100 g/m² and 200 g/m², in particular between 145 g/m² and 155 g/m². Alternatively, the adhesive of the attachment layer comprises or consists of acrylic adhesive. In this alternative embodiment, the adhesive of the attachment layer has a mass density between 8 g/m² and 50 g/m², and in particular close to 20 g/m². Such a density enables the patch to be sufficiently flexible and to adapt to all body types, and the receiving layer is sufficiently flexible to be able to mix the cosmetic composition and rub and thus increase the penetration of the cosmetic composition further.

According to another alternative embodiment, the layer for receiving a cosmetic composition is made of an occlusive material or of a breathable material.

An occlusive material is particularly adapted for a use of cosmetic compositions for quick treatment, by improving the effectiveness of the patch further.

An occlusive material is a material wherein the breathability is close to zero.

The breathability of a material is defined by its moisture vapor transmission rate (MVTR).

The MVTR is measured in g/m²/24 h.

The MVTR is tested as per the NF EN 13726-2 standard.

The higher the MVTR, the greater the breathability of the material, and the lower the occlusion.

A breathable material is particularly adapted for a use of cosmetic compositions for long-term treatment, by providing the user with additional comfort. According to an alternative embodiment, the blind cavity is split into a plurality of subcavities. The presence of a plurality of subcavities in the patch makes it possible to apply one or more cosmetic compositions according to the need, these cosmetic compositions optionally being for example an anti-wrinkle composition, a moisturizing composition, a decongestant composition, an acne treatment composition, etc.

According to other alternative embodiments,
the receiving layer delimits an outer peripheral edge, the intermediate layer including an outer peripheral edge having an identical contour to the peripheral edge of the receiving layer;
the cavity is sealed continuously at the periphery thereof by the intermediate layer. The present invention also relates to an assembly comprising a patch as defined above, and a storage sheet intended to receive the patch when the patch is not used.

The patch being repositionable, the storage sheet is used to protect the patch when it is not used from any dust and to maintain a good hold of the patch on the skin.

The present invention also relates to a kit comprising a patch as defined above and one or more cosmetic compositions capable of flowing, such as one or more liquid or pasty compositions or in gel or powder form, intended to be filled in the patch cavity or subcavities.

Such a kit makes it possible to produce extemporaneous mixtures of cosmetic compositions in the cavity or subcavities and that these compositions are mixed after fitting the patch thanks to the extremely malleable or flexible receiving layer.

Finally, this invention relates to a method for using a patch as defined above, comprising the following steps:
providing a patch as defined above;
filling the cavity or each of the subcavities with a cosmetic composition;
applying the patch on the skin with the cavity opening facing the skin;
attaching the patch to the skin by means of the attachment layer;
rubbing the receiving layer in order to distribute the cosmetic composition.

According to alternative embodiments,
the method comprises an initial step of detaching the patch from a storage sheet whereon the patch is initially attached by means of the attachment layer;
the method comprises the following additional steps:
detaching the patch from the skin;
washing the patch, in particular with water, so as to remove the cosmetic composition remaining in the cavity or subcavities;
attaching the patch to a storage sheet by means of the attachment layer.

The invention will be easier to understand after reading the following description, provided solely as an example, and with reference to the appended drawings, wherein:

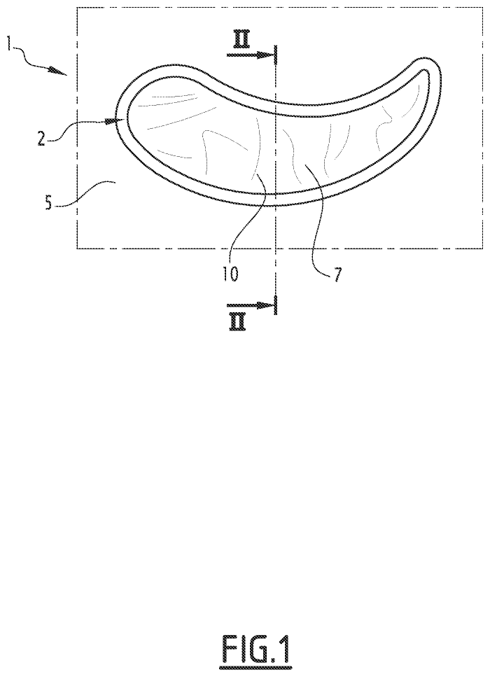

FIG. 1 is a top view of an embodiment example of a patch according to the invention placed on its storage sheet;

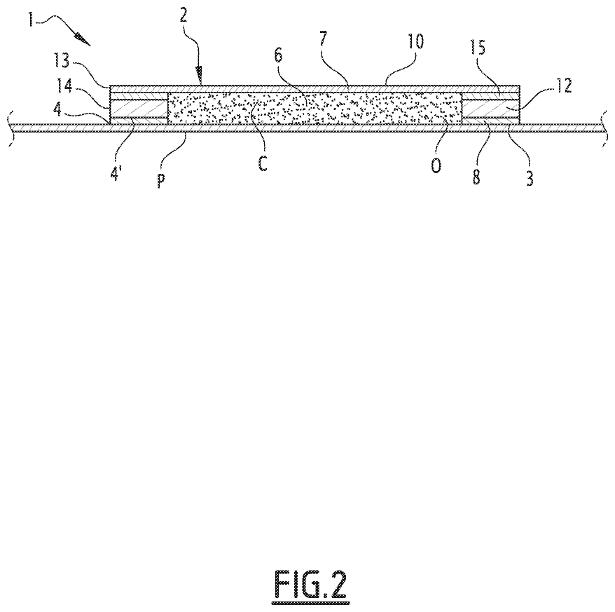

FIG. 2 is a schematic view of the patch in FIG. 1 as a cross-section along the plane II-II of FIG. 1 and applied to a users skin;

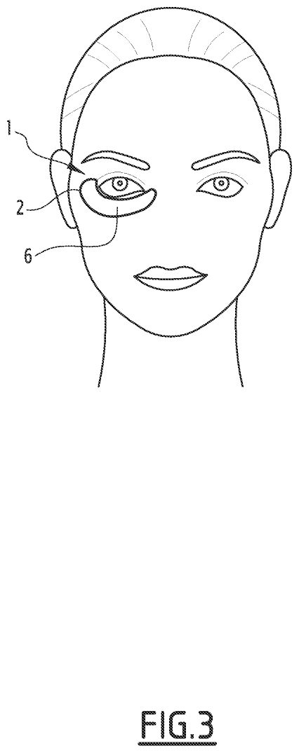

FIG. 3 is a schematic view of the patch in FIG. 1 on a users face; and

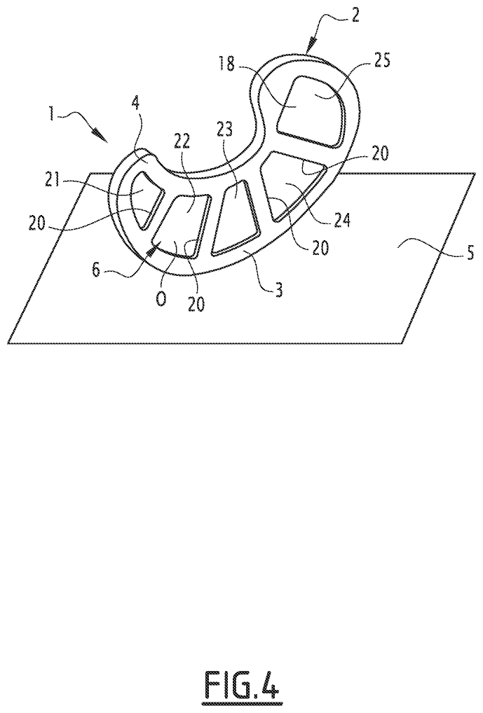

FIG. 4 is a schematic view of a patch according to a second embodiment example of the invention.

A patch 1 according to an embodiment example is represented in FIG. 1.

In FIG. 1, the patch 1 is adapted to be placed on a users face.

FIGS. 1 to 3 represent an embodiment example of a patch according to the invention, wherein the patch 1 is adapted to be placed below a users eyes to bring this area of the skin into contact with one or more cosmetic compositions.

More specifically, the patch 1 illustrated in the figures is adapted to bring one or more cosmetic compositions into contact with the area of a users skin located below their right eye.

As seen in particular in FIG. 1, the patch 1 comprises a substantially planar main body 2 and an attachment layer 4 on the skin, located at the periphery of the main body.

Substantially planar means that the main body 2 has first and second dimensions measured along two perpendicular axes, for example a length and a width, substantially greater, for example at least two times greater than the third dimension of the patch measured along a third axis perpendicular to the first two, for example a thickness.

The main body 2 consists of overlaid layers.

The main body 2 has an application face 3 adapted to be attached to a users skin by means of the attachment layer 4.

The attachment layer 4 comprises at least one adhesive.

Moreover, and as seen in FIG. 2, the attachment layer 4 of the patch 1 enables the attachment thereof on a storage sheet 5 when it is not used.

The storage sheet 5 is intended to be removed before the first use and protects the patch 1 from impurities.

The storage sheet 5 is advantageously made of polyester or of polyolefin.

According to the invention, the main body 2 of the patch 1 has at least one blind cavity 6 defining a bottom 7 and opening onto an opening O on the application face 3.

This cavity 6 is intended to directly receive a cosmetic composition C which is intended to be brought into contact with the skin area to be treated.

Moreover, the main body 2 of the patch 1 according to the invention has a peripheral attachment surface 8, formed on the application face 3, about the cavity 6.

The attachment layer 4 is applied on the peripheral attachment surface 8 of the application face 3 of the main body 2 and ensures the attachment of the patch 1 on the skin area to be treated.

According to an advantageous arrangement of the patch 1 according to the invention, the peripheral attachment surface 8 is closed onto itself, such that a seal is created around the blind cavity 6 between the patch 1 and the skin area to be treated, which makes it possible to keep the cosmetic composition C filled in the cavity 6 in contact with the skin for the entire duration of the treatment.

An occlusive space is thus defined by the patch 1 between the blind cavity 6 thereof and the skin area on which the patch 1 is applied, such an occlusive space helping improve the effectiveness of the skin treatment by means of the cosmetic composition C filled in the blind cavity 6 of the patch 1.

The occlusive space favors opening of the skin pores and accelerates the penetration of the cosmetic composition.

This also makes it possible to increase the quantity of cosmetic composition available for the treatment and thus the application time without the user being discommoded as the patch 1 is self-adhesive.

Moreover, the main body 2 is made of materials impervious to the cosmetic composition C.

Thus, when the patch 1 is placed on the skin P, the cosmetic composition C filled in the blind cavity 6 is confined in the space defined between the cavity 6 and the skin P, and can only diffuse in the direction of the skin area to be treated through the opening O of the cavity 6 placed facing this area.

Preferably, the attachment layer 4 is made of non-water-soluble materials, such that the patch 1 according to the invention can be reused after washing with water in order to remove the remaining cosmetic composition C filled in the cavity 6 of the patch 1 in the context of a previous application on the skin P, without the attachment layer 4 being detached from the main body 2 of the patch 1.

According to a first advantageous embodiment, the layer 4 includes a substrate coated with adhesive on both faces.

The substrate is for example a film made of polyurethane.

The substrate has for example a thickness of about 30 micrometers.

The attachment layer 4 of the patch 1 according to the invention is advantageously provided on the application face 3 with a pressure-sensitive adhesive.

Such a material for the attachment layer 4 is particularly advantageous as it enables the patch 1 according to the invention to be washable with water and reusable, the attachment layer 4 not losing the adhesion power thereof to the skin after washing. Moreover, such a material for the attachment layer 4 creates an enhanced seal between the patch 1 and the users skin.

The adhesive is advantageously a biocompatible adhesive intended to be applied on the skin.

“Biocompatible adhesive” denotes according to the invention any compound which has the property of adhering to a biological tissue when said compound is applied thereon, such as for example a mucous membrane or the dermis. In order to be biocompatible, the adhesive agent must be compatible with use on biological tissue, without causing adverse reactions such as inflammation of the biological tissue.

Preferably, the biocompatible adhesive comprises or consists of silicone, in particular a silicone gel.

Such an adhesive particularly ensures a satisfactory hold over time, as well as good repositionability of the cosmetic article on the skin.

Repositionability reflects the ability of the cosmetic article to be positioned, detached, then repositioned if needed to return it to its correct position.

For example, the biocompatible adhesive is selected from Silpuran® 2114, Silpuran® 2142, sold for example by WACKER, Silbione® 4743 or Silbione® 4645, sold for example by ELKEM, “Nero/black” silicone gel or other.

The adhesive of the attachment layer 4 on the application face 3, when it comprises or consists of silicone, in particular silicone gel, has a mass density between 20 g/m² and 300 g/m², preferably between 100 g/m² and 200 g/m², in particular between 145 g/m² and 155 g/m².

Preferably, the adhesive of the attachment layer 4 has a tack greater than or equal to 20 g/cm 2, preferably greater than or equal to 35 g/cm 2, more preferably greater than or equal to 50 g/cm 2, this tack being measured by means of the “probe tack” test.

For example, the adhesive of the attachment layer 4 has a tack between 50 g/cm 2 and 600 g/cm 2, preferably between 150 g/cm 2 and 500 g/cm 2, in particular between 350 g/cm 2 and 450 g/cm 2.

Moreover, the adhesive of the attachment layer 4 preferably has a hardness between 5 Shore A and 20 Shore A, preferably between 10 Shore 00 and 20 Shore 00.

Advantageously, the adhesive of the attachment layer 4 has a viscosity between 500 mPa·s and 3000 mPa·s, preferably between 1000 mPa·s and 2000 mPa·s.

According to another example, the adhesive of the attachment layer 4 on the application face 3 comprises or consists of biocompatible acrylic adhesive.

Such an adhesive ensures good repositionability of the cosmetic article on the skin.

The adhesive of the attachment layer 4 on the application face 3, when it comprises or consists of acrylic adhesive, has a mass density situated preferably between 8 g/m² and 50 g/m², and in particular close to 20 g/m².

Such an adhesive furthermore has a greater tackiness than the tackiness of the silicone gel, about ten times greater.

A superior tackiness gives rise to an enhanced holding capacity on the skin.

Acrylic adhesive furthermore has the advantage of reducing the bonding surfaces and thus of maximizing the space available for other functions of the patch 1 such as the cavity 6 while ensuring a good hold of the patch 1 on the skin P.

The main body 2 of the patch 1 comprises a layer 10 for receiving the cosmetic composition C.

The layer 10 for receiving the cosmetic composition C is intended to receive the cosmetic composition C.

The receiving layer 10 forms the bottom 7 of the patch 1.

The receiving layer 10 is thin enough for the user to be able to rub and thus increases the effectiveness of the treatment thanks to this mechanical effect.

The thickness of the receiving layer 10 is for example between 5 micrometers and 200 micrometers, in particular between 20 micrometers and 40 micrometers.

The receiving layer 10 is advantageously made of an occlusive material or of a breathable material.

The occlusive material is advantageously polyethylene (PE) or polypropylene (PP).

The breathable material is advantageously polyurethane.

According to this alternative embodiment, the breathable material is advantageously totally impervious to water.

The layer 10 is thus impervious to the cosmetic composition C.

The main body 2 of the patch 1 comprises an intermediate layer 12.

The intermediate layer 12 is disposed between the attachment layer 4 and the receiving layer 10.

Advantageously, the receiving layer 10 delimits an outer peripheral edge 13, the intermediate layer 12 includes an outer peripheral edge 14 having an identical contour to the peripheral edge 13 of the receiving layer 10.

The intermediate layer 12 is in the form of a strip closed onto itself defining at the center thereof at least a part of the cavity 6.

The cavity 6 is closed continuously at the periphery thereof by the intermediate layer 12. The thickness of the intermediate layer 12 determines the quantity of cosmetic composition C that the cavity 6 can contain. The thicker the intermediate layer 12, the greater the usable quantity of cosmetic composition C.

The intermediate layer 12 is made of a material absorbing the deformation in a transverse direction to the thickness of the patch 1.

The intermediate layer 12 provides the user with comfort and the patch 1 with flexibility.

The intermediate layer 12 advantageously comprises a foam. The foam is preferably made of polyethylene.

The thickness of the intermediate layer 12 is for example between 0.3 mm and 2.5 mm, preferably close to 0.5 mm.

According to the first embodiment of the attachment layer 4, said attachment layer 4 includes an adhesive on the face 4′ thereof in contact with the intermediate layer 12.

The adhesive on the face 4′ in contact with the intermediate layer 12 is for example an acrylic adhesive.

The adhesive on the face 4′ in contact with the intermediate layer 12 advantageously has a mass density between 30 g/m² and 50 g/m², in particular close to 40 g/m².

The adhesive on the face 4′ in contact with the intermediate layer 12 has a greater adherence than the adherence of the attachment layer 4 on the application face 3 thereof opposite the intermediate layer 12. Indeed, it is important that the attachment layer 4 adheres to the patch 1 and not to the skin P when the patch 1 is removed.

The adherence is for example characterized by the tack.

The adhesive present on the face 4′ in contact with the intermediate layer 12 makes it possible to protect the intermediate layer 12, in particular so that the adhesive on the application face 3 thereof opposite the intermediate layer 12 does not penetrate the cells of the foam when the intermediate layer 12 is made of foam.

According to a second embodiment of the attachment layer 4, said attachment layer 4 is a monolayer of adhesive.

Said adhesive is advantageously a biocompatible adhesive as described above.

Said adhesive has a mass density between 8 g/m² and 300 g/m². In particular, when the adhesive of the attachment layer 4 on the application face 3 comprises or consists of silicone, in particular silicone gel, said adhesive has a mass density preferably between 100 g/m² and 200 g/m², and in particular between 145 g/m² and 155 g/m². Alternatively, when the adhesive of the attachment layer 4 on the application face 3 comprises or consists of acrylic adhesive, said adhesive has a mass density preferably between 8 g/m² and 50 g/m², and in particular close to 20 g/m².

The main body 2 of the patch 1 further comprises a binding layer 15 sandwiched between the receiving layer 10 and the intermediate layer 12.

The binding layer 15 is in the form of a strip closed onto itself defining at the center thereof at least a part of the cavity 6. The outer contour and the inner contour of the binding layer 15 are disposed respectively with the outer contour and with the inner contour of the intermediate layer 12.

The binding layer 15 advantageously comprises a film provided with an adhesive on both faces. The adhesive of the binding layer 15 is for example an acrylic adhesive.

The film of the binding layer 15 is for example made of polyurethane (PU) and has for example a thickness of about 30 μm.

Advantageously, the adhesive of the binding layer 15 has a density close to 40 g/m².

Such an embodiment of the binding layer 15 facilitates industrial manufacture and reduces the manufacturing costs of the patch 1.

Alternatively, the binding layer 15 is a monolayer of adhesive.

The adhesive of the binding layer 15 is for example an acrylic adhesive.

The binding layer 15 makes it possible to hold the receiving layer 10 and the intermediate layer 12 together.

According to an advantageous arrangement of the patch 1 according to the invention, the volume of the cavity 6 is greater than or equal to 0.1 mL, preferably greater than or equal to 0.2 mL, more preferably greater than or equal to 0.5 mL and for example close to 1 mL.

The volume of the cavity 6 can however be greater than 1 mL, being for example of the order of 3 mL or more, in particular when the area of the skin on which it is intended to apply the patch 1 has a large surface area.

The thickness of the cavity 6 is for example between 0.2 mm and 3 mm, in particular close to 0.5 mm.

Thus the cavity 6 of the patch 1 according to the invention is capable of receiving a sufficient quantity of cosmetic composition C so that the latter is in contact with the users skin P for the entire desired treatment time.

The patch 1 can be presented in very varied shapes. For example, the main body 2 has a shape adapted to the treatment of an area of the skin corresponding to the eye contour, the lips, the forehead or to an area of the skin of restricted dimensions, for example for the localized treatment of an acne spot.

According to an alternative embodiment represented in FIG. 4, the cavity 6 is split into a plurality of subcavities 18 separated by partitions 20. In the specific example of a patch 1 to be placed below an eye, a first subcavity 21 of the patch, located in the vicinity of the inside corner of the users right eye when the patch 1 is positioned below their right eye can advantageously be filled with a decongestant cosmetic product, three adjacent subcavities 22, 23, 24, located substantially plumb with the users right eye can advantageously be filled with a cosmetic under-eye concealer composition, and a final subcavity 25, located in the vicinity of the outside corner of the users right eye can advantageously be filled with a cosmetic anti-wrinkle composition.

According to a second aspect, the invention proposes a method for using a patch 1 as described above.

In an initial step of the method according to the invention, the patch 1 being provided in the initial state by being attached to a storage sheet 5 illustrated in FIG. 1, the user detaches the patch 1 from the storage sheet 5 to which the patch 1 is initially attached by means of the attachment layer 4.

Then, the user fills the cavity 3 with a cosmetic composition C.

Then, the user applies the patch 1 to the area of the skin P to be treated, in this case below their right eye, as represented in FIG. 3, with the opening O of the cavity 3, facing the skin P, and attaches the patch 1 by means of the attachment layer 4 by applying a certain pressure, and more specifically a certain force, of the order of a few Newtons, thereon such that the attachment layer 4 adheres to the skin P.

The user then rubs the receiving layer 10 in order to distribute the cosmetic composition C on the area of the skin P to be treated.

Once the desired treatment time has elapsed, the user detaches the patch 1 from the skin P and can then wash it, in particular with water, so as to remove the cosmetic composition C in the cavity.

The attachment layer 4 retaining the adhesion power thereof even after cleaning with water, the user can then attach the patch 1 on the storage sheet 5 by means of the attachment layer 4.

The assembly formed of the patch 1 according to the invention and its storage sheet 5 can then advantageously be stored in a hermetic box until the next use of the patch 1, such that the patch 1 is not exposed to dust capable of fouling the attachment layer 4 and thus impairing the subsequent good hold of the patch 1 on the skin P.

In the embodiment where the biocompatible adhesive of the attachment layer 4 comprises or consists of acrylic adhesive, the patch 1 cannot be washed and reused.

Advantageously, to produce the patch 1, the receiving layer 10 is cut to the desired shape. According to the alternative embodiment wherein the binding layer 15 comprises a film provided with an adhesive on both faces, the binding layer 15 is cut to the desired shape then is coated with adhesive and is mounted by deposition on the receiving layer 10.

According to the alternative embodiment wherein the binding layer 15 is a monolayer of adhesive, it is applied on the receiving layer 10.

The intermediate layer 12 is cut to the desired shape then is mounted by deposition on the binding layer 15.

According to the alternative embodiment wherein the attachment layer 4 comprises a substrate coated with adhesive on both faces, the substrate of the attachment layer 4 is cut to the desired shape then is provided with an adhesive on the application face thereof and an adhesive on the other face 4′ thereof intended to be in contact with the intermediate layer 12, then is mounted by deposition on the intermediate layer 12.

According to the alternative embodiment wherein the attachment layer 4 is a monolayer of adhesive, the attachment layer 4 is cut to the desired shape then is mounted by deposition on the intermediate layer 12.

According to an alternative embodiment of the patch 1, and in particular of a plurality of patches, the binding layer 15, the intermediate layer 12 and the attachment layer 4 can be overlaid, in particular in sheet form, and bound together by means of the different adhesives described above, then cut together so as to define the opening O of the patch 1. The subassembly thus formed, presented in particular in the form of a stack of sheets perforated with a plurality of openings O, is then mounted on the receiving layer 10, in particular also in sheet form, and bound to the latter by means of the adhesive of the binding layer 15. A final cutting operation of the assembly thus formed makes it possible to define the outer contour of the patch 1, and in particular of a plurality of patches 1, to the desired shape. According to this alternative embodiment, particularly advantageous in terms of industrial embodiment, a plurality of patches 1 can be formed simultaneously by simultaneous cutting operations of the openings O and the outer contours, for example by means of dies provided with a plurality of punches of adapted shapes and arranged in relation to one another so as to minimize material offcuts.

The patch(es) 1 obtained by means of the different alternative process embodiments described above are then placed, individually or in batches, on a storage sheet 5, optionally cut or pre-cut subsequently, in order to form sets of patches 1 on individual or detachable storage sheets 5, ready for use.

The method can be carried out automatically and at a very high speed.

Although the patch according to the invention has been described in relation to an application to the treatment of the area under an eye, such an application is not restrictive and the patch according to the invention can absolutely be used on other areas of a users skin, for example, the forehead or the cheek, in particular for the localized treatment of an acne spot.

1. A patch for bringing a cosmetic composition into contact with the skin, comprising a substantially planar main body, having an application face adapted to be attached to the skin in a repositionable fashion by means of an attachment layer comprising at least one adhesive, the main body having a blind cavity defining a bottom and opening via an opening onto the application face, said cavity being adapted to directly receive the cosmetic composition to be brought into contact with the skin, and a peripheral attachment surface being formed on the application face about the cavity, the attachment layer extending over the peripheral attachment surface of the application face of the main body,
wherein the main body consists of overlaid layers, the overlaid layers comprising:
at least one layer for receiving a cosmetic composition, the receiving layer delimiting the bottom of the cavity,
at least one intermediate layer inserted between the layer for receiving a cosmetic composition and the attachment layer, the intermediate layer delimiting the periphery of the cavity, and defining, at the periphery of the cavity, the application face receiving the attachment layer.

2. The patch according to claim 1, wherein the main body further comprises a binding layer inserted between the intermediate layer and the receiving layer.

3. The patch according to claim 1, wherein the intermediate layer is made of foam.

4. The patch according to claim 1, wherein the attachment layer comprises a biocompatible adhesive intended to be applied on the skin.

5. The patch according to claim 1, wherein the attachment layer comprises a biocompatible adhesive intended to be applied on the skin, wherein the attachment layer includes an adhesive on the face thereof in contact with the intermediate layer, the adhesive having a greater adherence than the adherence of the attachment layer on the application face thereof opposite the intermediate layer.

6. The patch according to claim 1, wherein the attachment layer comprises a biocompatible adhesive intended to be applied on the skin, wherein the attachment layer includes an adhesive on the face thereof in contact with the intermediate layer, the adhesive having a greater adherence than the adherence of the attachment layer on the application face thereof opposite the intermediate layer, wherein at least one adhesive of the attachment layer has a mass density between 8 g/m² and 300 g/m².

7. The patch according to claim 1, wherein the attachment layer comprises a biocompatible adhesive intended to be applied on the skin, wherein the attachment layer includes an adhesive on the face thereof in contact with the intermediate layer, the adhesive having a greater adherence than the adherence of the attachment layer on the application face thereof opposite the intermediate layer, wherein the layer for receiving a cosmetic composition is made of an occlusive material or of a breathable material.

8. The patch according to claim 1, wherein the blind cavity is split into a plurality of subcavities.

9. The patch according to claim 1, wherein the receiving layer delimits an outer peripheral edge, the intermediate layer including an outer peripheral edge having an identical contour to the peripheral edge of the receiving layer.

10. The patch according to claim 1, wherein the cavity is sealed continuously at the periphery thereof by the intermediate layer.

11. An assembly comprising a patch according to claim 1, and a storage sheet intended to receive the patch when the patch is not used.

12. A kit comprising a patch according to claim 1, and one or more cosmetic compositions capable of flowing, such as one or more liquid or pasty compositions or in gel or powder form, intended to be filled in the cavity or subcavities of the patch.

13. A method for using a patch according to claim 1, comprising the following steps:
providing a patch according to claim 1;
filling the cavity or each of the subcavities with a cosmetic composition;
applying the patch on the skin with the opening of the cavity facing the skin;
attaching the patch to the skin by means of the attachment layer;
rubbing the receiving layer in order to distribute the cosmetic composition.

14. The method according to claim 13, comprising an initial step of detaching the patch from a storage sheet whereon the patch is initially attached by means of the attachment layer.

15. The method according to claim 13, comprising the following additional steps:
detaching the patch from the skin;
washing the patch, in particular with water, so as to remove the cosmetic composition remaining in the cavity or subcavities;
attaching the patch to a storage sheet by means of the attachment layer.

16. The patch according to claim 2, wherein the intermediate layer is made of foam.

17. The patch according to claim 2, wherein the attachment layer comprises a biocompatible adhesive intended to be applied on the skin.

18. The patch according to claim 3, wherein the attachment layer comprises a biocompatible adhesive intended to be applied on the skin.

19. The patch according to claim 2, wherein the attachment layer comprises a biocompatible adhesive intended to be applied on the skin, wherein the attachment layer includes an adhesive on the face thereof in contact with the intermediate layer, the adhesive having a greater adherence than the adherence of the attachment layer on the application face thereof opposite the intermediate layer.

20. The patch according to claim 3, wherein the attachment layer comprises a biocompatible adhesive intended to be applied on the skin, wherein the attachment layer includes an adhesive on the face thereof in contact with the intermediate layer, the adhesive having a greater adherence than the adherence of the attachment layer on the application face thereof opposite the intermediate layer.
